(19) United States
(12) Patent Application Publication
(10) Pub. No.: US 2016/0074187 A1
(43) Pub. Date: Mar. 17, 2016

(54) CATHETER SYSTEM WITH EXPANDABLE AND DELAMINATING TIP PORTION AND METHOD FOR PRODUCING SAME

(71) Applicant: BIOTRONIK AG, Buelach (CH)

(72) Inventor: Bodo Quint, Dettighofen (DE)

(21) Appl. No.: 14/827,871

(22) Filed: Aug. 17, 2015

Related U.S. Application Data
(60) Provisional application No. 62/050,212, filed on Sep. 15, 2014.
(60) Provisional application No. 62/050,211, filed on Sep. 15, 2014.

(51) Int. Cl. A61F 2/962; A61F 2/82

(52) U.S. Cl. CPC A61F 2/962; A61F 2/82

(57) ABSTRACT

A catheter system with a distal functional element, such as a self-expanding stent, includes a feed tube. The distal end of the feed tube surrounds the functional element and a sliding element configured to strike against the proximal end of the functional element in order to push forward the functional element from the feed tube. The feed tube has a tip portion that is tapered in the delivered state and expands when the functional element is slid out. The tip portion is formed from a glued e-PTFE laminate that delaminates when stretched by the functional element.

### PRIORITY CLAIM

This application claims priority under 35 U.S.C. §119 from prior U.S. Provisional Application No. 62/050,211, filed Sep. 15, 2014 and from prior U.S. Provisional Application No. 62/050,212, filed Sep. 15, 2014.

### FIELD OF THE INVENTION

The field of the invention is catheter systems that have a functional element at the distal end.

### BACKGROUND

An example functional element is a self-expanding stent. However, the invention is generally suitable for catheter systems in which a functional element is surrounded by a feed tube and is pushed out from the feed tube with the aid of a sliding element, often referred to as a pusher. Other example functional elements are used in mechanically acting catheter systems that are designed to remove tissue samples, to remove tissue deposits or to penetrate tissue.

Within the scope of this application, the distal end, for example of the catheter system, is understood to be the end that is remote from the user. The proximal end of the catheter system is accordingly closest to the user.

Self-expanding stents that function without a balloon catheter are known and are in widespread clinical use. In view of the preferred used materials for production, such stents are also referred to for example as “nitinol stents”; they are offered commercially by the present applicant, inter alia.

Known catheter systems for self-expanding stents include systems without any “inner lumen.” These systems can be produced advantageously with small diameter and enable a reduction of the trauma in the case of catheter intervention. These catheter systems also offer particularly high flexibility. In such systems, the stent itself forms the lumen for the guide wire, and the distal catheter end (tip) is connected to the feed tube or formed as a distal portion thereof. The tip is opened via plastic deformation when the stent is slid out distally from the feed tube by the sliding element. The mechanical properties of the tip have to satisfy both the requirements when sliding out the stent (release) and when withdrawing the catheter.

Such systems consist only of the feed tube, which surrounds the self-expanding stent, the sliding element and the guide wire. The self-expanding stent is held in the compressed (“crimped”) state thereof by the feed tube. The stent is slid out from the feed tube by the sliding element and is expanded (“dilated”) into the surrounding blood vessel in order to widen or support said blood vessel. Such systems do away with the otherwise usual inner tube or inner shaft, which forms the guide wire lumen or surrounds the guide wire and carries the self-expanding stent.

Such catheter systems without an inner shaft accordingly have the advantage that they can be reduced in diameter, whereby a greater flexibility is achieved in general and smaller auxiliary elements (insertion aids and/or guide catheters) can be used.

In these systems, the distal catheter tip must be fixed to the feed tube. The catheter tip rounds off the feed tube and ensures that the tissue is not traumatized or damaged as the catheter system is introduced and advanced to the treatment site in the body at which the functional element is to perform the function thereof. During the deformation by the release of the functional element, the tip should not be plastically deformed in such a way that it is potentially traumatic following deformation or hinders the withdrawal of the catheter system from the body. A resilient deformation is ideal, to avoid trauma and to release the functional element under constant friction due to the resilient restoring force of the catheter tip.

### SUMMARY OF THE INVENTION

A preferred embodiment is catheter system with a distal functional element, such as a self-expanding stent, that includes a feed tube. The distal end of the feed tube surrounds the functional element and a sliding element configured to strike against the proximal end of the functional element in order to push forward the functional element from the feed tube. The feed tube has a tip portion that is tapered in the delivered state and expands when the functional element is slid out. The tip portion is preferably formed from a glued e-PTFE laminate that delaminates when stretched by the functional element.

A preferred embodiment is method for producing a catheter system with a feed tube that has a tip portion that is expandable and delaminates when stretched by the functional element. The method forms the tip portion of the feed tube from an e-PTFE laminate pre-configured in a tubular manner by impregnating the laminate with an alcohol solution containing a fatty acid at a first increased temperature that is elevated above room temperature and subsequently molding of the impregnated tip portion at a second increased temperature.

### BRIEF DESCRIPTION OF THE DRAWINGS

In the figures

FIG. 1 shows an exemplary embodiment according to the invention of a catheter system for implantation of a self-expanding stent, and

FIG. 2a-f shows an exemplary embodiment for producing and fastening a tip on to a feed tube according to the invention.

### DETAILED DESCRIPTION OF THE PREFERRED EMBODIMENTS

The inventor has determined that for a catheter system without an inner tube, the tip should have different geometric configurations and mechanical properties in various phases of use: In the delivered state the diameter of the system (especially of the feed tube) should reduce toward the distal end, that is to say a type of tip should actually be formed, which additionally has a sufficient rigidity. In the feed phase, this tip should be deformable and should ideally have a second geometric configuration, for example should be cylindrical or trumpet-shaped, and should then be relatively soft and exert minimal restoring forces onto the functional element to be released. So as also not to negatively influence an atraumatic return of the catheter system or withdrawal thereof by potential auxiliary systems, the mechanical behavior of the deformed region should also change. The mechanical properties of the undeformed tip should be characterized by toughness and flexibility, whereas the deformed tip is to have a considerably increased flexibility and a minimal rigidity compared to the undeformed tips. In both states, the mechanical integrity of the tip must be retained. The surface is to be low-friction so as not to irritate the bodily vessel upon contact therewith.

Preferred methods of forming the distal end portion of the system form a tip that is stiff during introduction and during the feed or the release of the functional element, such as during the expansion phase of a stent. The release of the stent or other functional element reduces the stiffness of the tip configuration and the mechanical rigidity, which are largely cancelled due to a predetermined material conversion. Preferred embodiments leverage an e-PTFE layer material to form the tip.

E-PTFE, a form of polytetrafluoroethylene, is known to be a very soft, resilient, low-friction and hydrophobic material. Properties can be adjusted very well by the thermal production method to resilient, plastic and strength requirements. The noteworthy flexibility in comparison to strength is based on the microstructure of e-PTFE produced during sintering and warping processes in the production process.

This microstructure, in contrast to conventional PTFE components, enables excellent bonding capacity when the microstructure of the e-PTFE can be penetrated by an adhesive. Methods of the invention produce a reliable connection of an e-PTFE tube to a connecting tube, particularly a “release tube” or feed tube” of a catheter.

In preferred embodiment catheter systems, the distal end portion of the feed tube consists of an e-PTFE laminate in a tip configuration that is temporarily glued in such a way that the glued bond, which is both brought into its shape and also mechanically stiffened by the gluing, delaminates at least in part in the feed phase (where it is subject to stretching). As a result of this delamination, the formed geometric shape is lost at least gradually, that is to say the original tip widens and the desirable cylinder or trumpet shape mentioned further above is formed. Furthermore, the mechanical rigidity is lost at least in part, such that the desired increased softness and reduced restoring forces acting on the stent arise.

In one embodiment of the invention, the tip portion of the feed tube is in the shape of a truncated cone or is molded with a rounded distal end. Here, the distal end may be formed spherically, and a conical tip may especially have a spherical termination.

In accordance with an embodiment of the material, the e-PTFE laminate is glued using an adhesive that has a fatty acid, preferably stearic acid, as a component essential to function. Here, in accordance with embodiments, the adhesive comprises an oleic acid additive or strength-increasing polymeric additives such as polyvinylpyrrolidone or the like. The combination of stearic acid with oleic acid is to be understood only as a preferred example for expedient combinations of adhesive components for the temporary gluing; in addition, other polymers soluble in the fatty acid matrix apart from PVP can contribute to the property improvement of the fatty acid essential to function.

In a further embodiment of the invention, the catheter system comprises a stent, which is formed in such a way that it acts as a solid under shear load as it is advanced from the feed tube. The term “acts as a solid” is to be understood within the scope of this application to mean that the stent is configured in such a way that individual stent segments do not overlap or are not slid over one another or canted against one another. Among the large number of known and, for the most part, also commercially available stent constructions, a person skilled in the art can find those that meet this requirement and can thus advantageously cooperate with the proposed design of the distal end region of the system.

In preferred geometric configurations, the length of the tip portion is in the range of 0.3 times to 5 times, in particular 0.6 times to 2 times, the outer diameter of the feed tube. However, it is to be understood that configurations also outside these relations, in which the mentioned temporary gluing of the end portions that can be destroyed by stretching load is present, also belong to the invention.

In a further embodiment, the tip portion is formed integrally with the feed tube. This embodiment is particularly easily produced technically and also can be subjected to strong mechanical load. However, a bond between a tip made of a first material and provided with temporary gluing and a feed tube made of a second material can also be considered as covered by the scope of the invention in principle. With such an alternative embodiment, the materials for the long feed tube and the short end portion can be selected in a differentiated manner and adapted to the respective requirements.

Conventional methods are basically suitable for production of the present catheter system apart from the tip portion, wherein the production of the temporary gluing of the feed tube end portions rescindable by stretching in accordance with a preferred embodiment. Depending on the material selection of the adhesive and depending on whether or not the end portion is formed integrally with the other feed tube, a large number of method variants are possible in principle.

Especially for the use of fatty acids as a component of the adhesive bond essential to function, a method approach is provided in which an e-PTFE laminate or e-PTFE component (formed in a number of layers) pre-configured in a tubular manner is impregnated with a solution containing the fatty acid (especially stearic acid) at elevated temperature and is then dried. Later, the desired geometric shape of the tip is formed by molding, likewise at increased temperature, and at the same time causes a compression and thus mechanical compaction of the material structure. Following the cooling, a tip portion having the properties desired for the delivered situation of the application system is provided.

In embodiments of this method, the first increased method temperature preferably lies in the range between 60° C. and 90° C., more preferably between 70° C. and 85° C. and most preferably at 80° C. and the second method temperature is above the melting point of the fatty acid, is preferably in the range between 70° C. and 90° C. and most preferably in the range between 75° C. and 85° C.

A special advantage of this application method is based on the observation that a crystalline precipitate of the stearic acid is preferably obtained, dried from solution. If the e-PTFE is impregnated with this solution, it initially still remains relatively flexible. Under pressure and compression, such an impregnated tube can be compressed in the form of a conical tip. From a temperature starting at the melting point (69° C.), the stearic acid is melted and in principle forms a “fiber composite material” in conjunction with the e-PTFE, which fiber composite material is at least very resistant to compression and demonstrates low friction. Under tensile load, however, due to the low strength of the matrix, a delamination of the matrix then occurs, and the “e-PTFE” tube is released again depending on the load. This tube then recovers its flexibility.

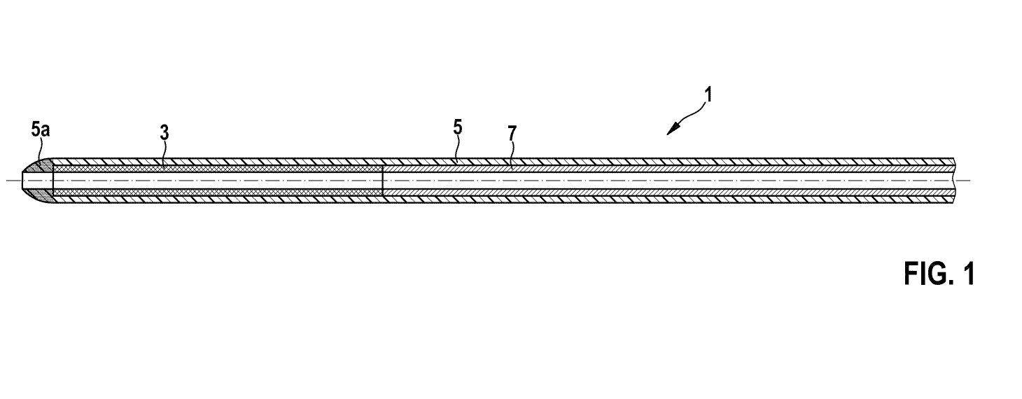

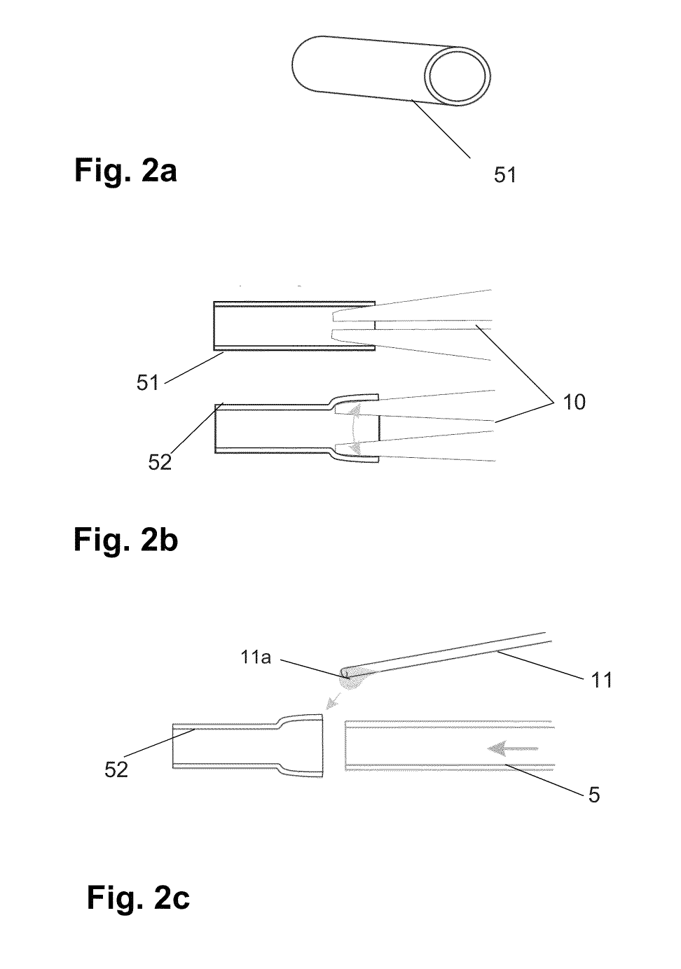

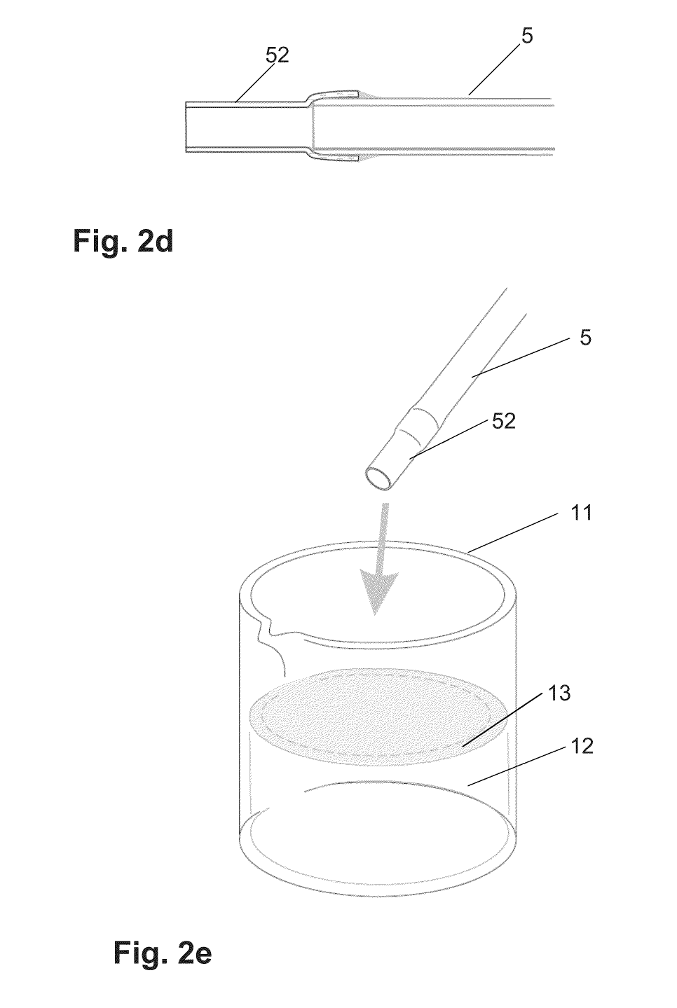

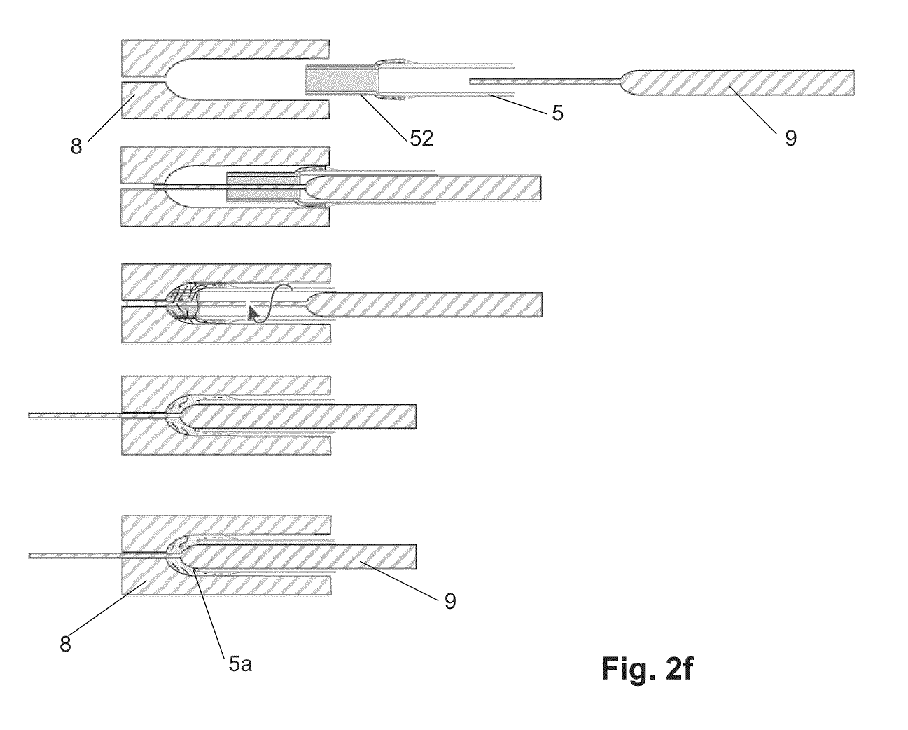

Advantages and expedient features of the invention will emerge from the following description on the basis of the exemplary embodiment of the invention illustrated in FIG. 1. FIGS. 2a-2f show an exemplary embodiment of a method for producing a feed tube having such a tip.

FIG. 1 schematically shows the distal end region of a catheter system 1 of a self-expanding stent 3. The stent 3 is preferably made of nitinol and, in the delivered condition shown in the figure, is housed in a compressed manner in a distal end portion of a feed tube 5, in which a flexible pushing rod 7 is additionally housed as a sliding element for releasing the stent and strikes proximally against the proximal end of the stent. The feed tube 5 is formed from an e-PTFE laminate and a temporarily glued, compressed tip region running in a chronically curved manner is formed in a distal end portion 5a by impregnation with an adhesive based on stearic acid and thermal compression treatment in the above-described manner.

This tip region is relatively stiff in the delivered state. As the stent 3 is advanced by the pushing rod 7, the tip region is stretched by the stent, and the adhesive matrix is broken and the material bond is delaminated in this respect and is reconfigured into a cylindrical to trumpet-shaped release state, which is also mechanically much softer compared with the starting state. The high slidability of the e-PTFE additionally facilitates the advance of the stent as well as the withdrawal of the feed tube, once said feed tube has been fed, from the corresponding vessel.

FIGS. 2a to 2f show an exemplary embodiment of the production of a feed tube according to the invention for a catheter system according to the invention. FIG. 2a shows a tailor-cut piece 51 of an e-PTFE tube, which, as shown in FIG. 2b, is widened using a suitable tool 10 (for example a pair of tongs) at one end. The widened e-PTFE tube piece 52 is contacted by means of a suitable bar 11 with a cyanoacrylate adhesive 11a and is slid onto the end of a feed tube 5 (FIGS. 2e, 2d), such that a feed tube tip preform as in FIG. 2d is produced. This preform formed of e-PTFE tube piece 52 and feed tube 5 is dipped into a vessel 12 containing a solution 13 containing stearic acid (FIG. 2e), such that the e-PTFE tube piece 52 is coated with this solution. The combination of coated e-PTFE tube piece 52 and feed tube 5 is slid using a suitable plunger 9 into a heatable mold 8 (FIG. 20. In the mold 8, the combination of e-PTFE tube piece 52 and feed tube 5 is heated with movement up to above the melting point of the olefin matrix (to above 76° C. in this embodiment) and is pressed together (compacted). Following subsequent cooling, a feed tube 5 according to the invention is thus created, with a distal end portion 5a which is formed as a tip within the sense of the invention.

The embodiment of the invention is not limited to the above-described examples and emphasized aspects, but a large number of modifications are also possible within the capabilities of a person skilled in the art.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A catheter system comprising:
a feed tube,
a functional element surrounded by a distal end of the feed tube,
a sliding element configured to strike against a proximal end of the functional element in order to push forward the functional element from the feed tube, and
a tip portion at the distal end of the feed tube that is tapered in a delivered state and is expandable when the functional element is slid out, the tip portion comprising a glued e-PTFE laminate that delaminates when stretched by the functional element.

2. The catheter system according to claim 1, wherein the functional element is a self-expanding stent.

3. The catheter system according to claim 1, wherein the tip portion of the feed tube is shaped as a truncated cone or is molded with a rounded distal end.

4. The catheter system according to claim 1, wherein the tip portion of the feed tube is molded with a rounded distal end.

5. The catheter system according to claim 1, wherein the e-PTFE laminate is glued by an adhesive that comprises a fatty acid.

6. The catheter system according to claim 5, wherein the fatty acid comprises stearic acid.

7. The catheter system according to claim 5, wherein the adhesive comprises an oleic acid- or strength-increasing polymeric additive of polyvinylpyrrolidone.

8. The catheter system according to claim 1, wherein the length of the tip portion is in the range of 0.3 times to 5 times the outer diameter of the feed tube.

9. The catheter system according to claim 8, wherein the length of the tip portion is in the range of 0.6 times to 2 times the outer diameter of the feed tube.

10. The catheter system according to claim 1, wherein the tip portion is formed integrally with the feed tube.

11. A method for producing a catheter system with a feed tube that has a tip portion that is expandable and delaminates when stretched by the functional element, the method comprising:
forming the tip portion of the feed tube from an e-PTFE laminate pre-configured in a tubular manner by impregnating the laminate with an alcohol solution containing a fatty acid at a first increased temperature that is elevated above room temperature and subsequent molding of the impregnated tip portion at a second increased temperature.

12. The method according to claim 11, wherein the fatty acid comprises stearic acid.

13. The method according to claim 11, wherein the first increased temperature lies in the range between 60° C. and 90° C. and the second increased temperature is above the melting point of the fatty acid.

14. The method according to claim 13, wherein the first increased temperature lies in the range between 70° C. and 85° C. and the second increased temperature is in the range between 70° C. and 90° C.

15. The method according to claim 14, wherein the first increased temperature is 80° C. and the second method temperature is in the range between 75° C. and 85° C.
